(19) United States
(12) Patent Application Publication
(10) Pub. No.: US 2014/0073930 A1
(43) Pub. Date: Mar. 13, 2014

(54) MEASURE OF BRAIN VASCULATURE COMPLIANCE AS A MEASURE OF AUTOREGULATION

(71) Applicants: Rakesh Sethi, Vancouver (CA); James N. Watson, Dunfermline (GB); Paul S. Addison, Edinburgh (GB)

(72) Inventors: Rakesh Sethi, Vancouver (CA); James N. Watson, Dunfermline (GB); Paul S. Addison, Edinburgh (GB)

(73) Assignee: NELLCOR PURITAN BENNETT LLC, Boulder, CO (US)

(21) Appl. No.: 13/606,301

(22) Filed: Sep. 7, 2012

(51) Int. Cl. A61B 5/021; A61B 6/00

(52) U.S. Cl. 600/473; 600/485

(57) ABSTRACT

A system includes a controller that receives a physiological signal representing a non-invasive measure of a physiological parameter. The controller applies a compliance metric to the physiological signal and generates an autoregulation status signal that indicates a status of cerebral autoregulation in the patient. The autoregulation status signal is based at least in part on the compliance metric applied to the physiological signal.

### BACKGROUND

Cerebral blood flowsupplies oxygen and nutrients to the brain. A drop in blood flow can cause ischemia which may result in tissue damage or death of brain cells. An increase in blood flow can cause hyperminia which may result in swelling of the brain or edema. Autoregulation is a process that attempts to maintain an optimal blood flow to the brain.

Following a reduction in blood flow to the brain, the initial response of the body is peripheral vasoconstriction, which reduces blood flow to non-essential areas of the body while maintaining blood pressure. The secondary response is pressure autoregulation in the cerebral area. During autoregulation, cerebral arterioles dilate as cerebral pressure falls in the attempt to maintain blood flow. As cerebral pressure increases, cerebral arterioles constrict to reduce the blood flow that could also cause injuries.

### BRIEF DESCRIPTION OF THE DRAWINGS

FIG. 1 illustrates an exemplary system for non-invasively detecting cerebral autoregulation impairment.

FIG. 2 is a flowchart of an exemplary process that may be used by the system of FIG. 1.

FIG. 3 is a chart showing examples of pressure and velocity pulses through a more compliant and less compliant blood vessel.

FIG. 4 is a chart of an example ensemble average photoplethysmograph (PPG) derivative pulses and high and low arterial pressures.

FIG. 5 illustrates example arterial pressure with pulse derivative skew.

### DETAILED DESCRIPTION

An exemplary system includes a controller that receives a physiological signal. This physiological signal may be a plethysmographic signal, photondensity wave signal, photoacoustic signal, blood flow signal, blood pressure signal, etc. The controller applies a compliance metric to the physiological signal and generates an autoregulation status signal that indicates a status of cerebral autoregulation in the patient. One possible process implemented by the system includes the following: receiving a physiological signal representing a non-invasive blood pressure measurement, applying a compliance metric to the received physiological signal, and generating an autoregulation status signal that indicates a status of cerebral autoregulation in the patient. Again, the autoregulation status signal is based at least in part on the compliance metric applied to the physiological signal.

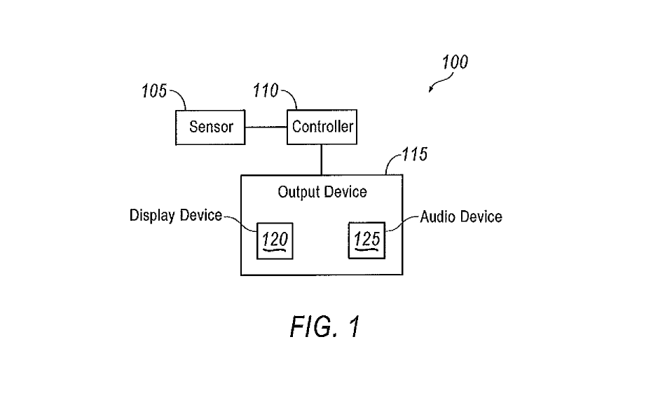

FIG. 1 illustrates an exemplary system 100 for detecting cerebral autoregulation impairment. As illustrated in FIG. 1, the system 100 includes a sensor 105, a controller 110, and an output device 115. The system 100 may take many different forms and include multiple and/or alternate components and facilities. While an exemplary system 100 is shown, the exemplary components illustrated in Figure are not intended to be limiting. Indeed, additional or alternative components and/or implementations may be used.

The sensor 105 may include any device configured to non-invasively measure a physiological parameter of a patient, such as the patient's blood pressure represented by a blood pressure signal. Other possible physiological parameters may be represented by a plethysmographic signal, photondensity wave signal, photoacoustic signal, blood flow signal, etc. In one exemplary approach, the sensor 105 may include a near-infrared spectroscopy sensor 105. That is, the sensor 105 may be configured to generate light in the near-infrared spectrum, from about 800 nm to about 2500 nm, and receive reflected light. The sensor 105 may in some circumstances process the reflected light at least to generate a representative signal. The sensor 105 may be configured to measure various physiological parameters including oxygen saturation, hemoglobin, blood pressure, etc. The representative signal, therefore, may indicate an amount of oxygen or hemoglobin in a patient's blood or a particular organ or other tissue. In operation, the sensor 105 may be placed on the patient to measure physiological parameters at the periphery (e.g., such as on the patient's finger) since changes at the periphery may indicate the initial states of an autonomic response to dropping regional oxygen saturation values, which may be a warning or the precursor that autoregulation is starting to fail.

The representative signal may, in some instances, represent the patient's blood pressure. Blood pressure may be defined as the pressure exerted on blood vessel walls, such as arterial or venous walls, during each heartbeat. The blood pressure may include a systolic value, which represents the patient's maximum blood pressure, and a diastolic value, which represents the patient's minimum blood pressure. Mean arterial pressure may represent the patient's average blood pressure during a cardiac cycle. The blood pressure signal generated by the sensor 105 may represent blood pressure values at various times. Other possible representative signals may include a plethysmographic signal, photondensity wave signal, photoacoustic signal, blood flow signal, etc.

The systolic and diastolic values of blood pressure may be based upon a property of the blood vessel called compliance. Compliance describes the ability of the blood vessel, such as a vein or artery, to resist recoil following a decrease in internal pressure. Compliance is the reciprocal of elasticity and may be defined as the change in volume of the vessel over the change in internal pressure of the vessel. A highly compliant blood vessel will deform easier than a blood vessel exhibiting lower compliance. Some vessels have the ability to change their compliance properties such as arterioles.

The controller 110 may include any device configured to receive and process representative signals, such as the blood pressure signal, from the sensor 105. The controller 110 may be configured to apply a compliance metric to the physiological signal. The compliance metric may include an equation or algorithm that, when applied to the physiological signal received from the sensor 105, may be used to determine a compliance value that represents the compliance of the blood vessel. The controller 110 may be configured to determine the compliance value by calculating at least one of the following characteristics of the measured physiological parameter: mean, standard deviation, skew, kurtosis, pulse wave area, center of area, rotational moment, pulse period, and peak to peak amplitude. Moreover, the measured physiological signal may be processed to facilitate determination of the compliance value. In some instances, the controller 110 may determine the compliance value from a first derivative of the physiological signal.

Moreover, the controller 110 may be configured to extract the blood pressure or other physiological parameter from the physiological signal. The compliance value and physiological parameter may be used to determine an autoregulation status of the patient. The autoregulation status may represent whether the cerebral autoregulation of the patient has become impaired. The controller 110 may be further configured to generate an autoregulation status signal that represents the determined autoregulation status of the patient. The autoregulation status signal may, in some exemplary approaches, be based at least in part on the compliance metric applied to the physiological signal received from the sensor 105. Specifically, the autoregulation status signal may be based on the compliance value determined by the controller 110.

The controller 110 may implement different methods to determine the autoregulation status of the patient from the compliance value. For instance, in one possible approach, the controller 110 may compare the compliance value to a plurality of known autoregulation parameters. Each autoregulation parameter may indicate whether, given a particular compliance value, cerebral autoregulation is impaired or working properly. The controller 110 may be configured to select the autoregulation parameter closest to the determined compliance value and base the autoregulation status, and associated autoregulation status signal, on the selected autoregulation parameter.

Alternatively, the controller 110 may be configured to compare the compliance value to a threshold autoregulation value. The threshold autoregulation value may represent, e.g., a minimum value indicating compliance. The controller 110 may be configured to determine compliance, and thus whether autoregulation is impaired, based on the compliance value relative to the threshold autoregulation value and generate the autoregulation status signal accordingly.

In some instances, the controller 110 may be further configured to generate an alarm signal if the autoregulation status indicates that cerebral autoregulation is impaired. The alarm signal may be transmitted with the autoregulation status signal or as a separate signal. That is, in some instances, the autoregulation status signal may only be generated if cerebral autoregulation is impaired, in which case the autoregulation status signal may also act as the alarm signal. In other instances where the autoregulation status signal is generated whether cerebral autoregulation is impaired or not, the alarm signal may be a separate signal. In any event, the alarm signal may be used, as described below, to generate a visual or audio representation of the status of cerebral autoregulation. For instance, if cerebral autoregulation is impaired, the alarm signal may cause the generation of various sounds or images designed to alert a treating physician's attention to the issue. Example visual representations may be particular colors, one or more flashing lights, the compliance value, a word description of the autoregulation status (e.g., the word “impaired”), etc. Example audio representations may include a buzzer, siren, alarm, or the like.

The output device 115 may include any device configured to receive the autoregulation status signal from the controller 110 and visually and/or audibly output information in accordance with the autoregulation status signal. For instance, the output device 115 may include a display device 120 configured to provide a visual representation of the status of cerebral autoregulation determined by the controller 110. Moreover or in the alternative, the output device 115 may include an audio device 125 configured to audibly provide sounds in accordance with the alarm signal, the autoregulation status signal, or both.

In general, computing systems and/or devices, such as the controller 110 and the output device 115, may employ any of a number of computer operating systems, including, but by no means limited to, versions and/or varieties of the Microsoft Windows® operating system, the Unix operating system (e.g., the Solaris® operating system distributed by Sun Microsystems of Menlo Park, Calif.), the AIX UNIX operating system distributed by International Business Machines of Armonk, N.Y., and the Linux operating system. Examples of computing devices include, without limitation, a computer workstation, a server, a desktop, notebook, laptop, or handheld computer, or some other known computing system and/or device.

Computing devices generally include computer-executable instructions, where the instructions may be executable by one or more computing devices such as those listed above. Computer-executable instructions may be compiled or interpreted from computer programs created using a variety of programming languages and/or technologies, including, without limitation, and either alone or in combination, Java™, C, C++, Visual Basic, Java Script, Perl, etc. In general, a processor (e.g., a microprocessor) receives instructions, e.g., from a memory, a computer-readable medium, etc., and executes these instructions, thereby performing one or more processes, including one or more of the processes described herein. Such instructions and other data may be stored and transmitted using a variety of known computer-readable media.

A computer-readable medium (also referred to as a processor-readable medium) includes any non-transitory (e.g., tangible) medium that participates in providing data (e.g., instructions) that may be read by a computer (e.g., by a processor of a computer). Such a medium may take many forms, including, but not limited to, non-volatile media and volatile media. Non-volatile media may include, for example, optical or magnetic disks and other persistent memory. Volatile media may include, for example, dynamic random access memory (DRAM), which typically constitutes a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, or any other medium from which a computer can read.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

Databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store is generally included within a computing device employing a computer operating system such as one of those mentioned above, and are accessed via a network in any one or more of a variety of manners, as is known. A file system may be accessible from a computer operating system, and may include files stored in various formats. An RDBMS generally employs the Structured Query Language (SQL) in addition to a language for creating, storing, editing, and executing stored procedures, such as the PL/SQL language.

In some examples, system elements may be implemented as computer-readable instructions (e.g., software) on one or more computing devices (e.g., servers, personal computers, etc.), stored on computer readable media associated therewith (e.g., disks, memories, etc.). A computer program product may comprise such instructions stored on computer readable media for carrying out the functions described herein.

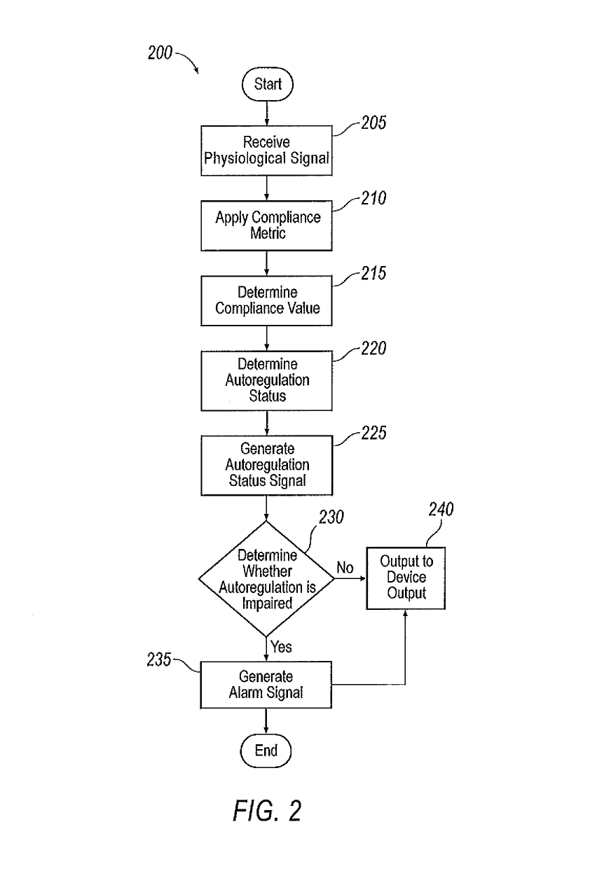

FIG. 2 illustrates a flow chart of an exemplary process 200 that may be implemented by the system 100 of FIG. 1 to, for example, non-invasively determine whether cerebral autoregulation of a patient has become impaired and to take an appropriate remedial measure.

At block 205, the controller 110 may receive the physiological signal from the sensor 105. The sensor 105, as discussed above, may include a near-infrared spectroscopy sensor 105 configured to non-invasively measure a physiological parameter of a patient, generate the physiological signal in accordance with the measured physiological parameter, and transmit the physiological signal to the controller 110. The physiological signal received by the controller 110, therefore, represents a non-invasive physiological parameter measurement.

At block 210, the controller 110 may apply the compliance metric to the received physiological signal. Applying the compliance metric may include applying an equation or algorithm to the physiological signal.

At block 215, the controller 110 may determine a compliance value. In one possible approach, determining the compliance value may occur after applying the compliance metric to the physiological signal. In some instances, the compliance value may be the direct result of applying the compliance metric to the physiological signal. Determining the compliance value includes may include calculating at least one of the following characteristics of the measured physiological parameter: mean, standard deviation, skew, kurtosis, pulse wave area, center of area, rotational moment, pulse period, and peak to peak amplitude.

At block 220, the controller 110 may determine the autoregulation status of the patient based on, e.g., the compliance value. For example, as previously discussed, the controller 110 may compare the compliance value to a plurality of known autoregulation parameters to determine the autoregulation status. Alternatively, the controller 110 may base the autoregulation status on, e.g., whether the compliance value exceeds a threshold autoregulation value. Using these or other methods, the controller 110 may determine whether the compliance value indicates that cerebral autoregulation is impaired.

At block 225, the controller 110 may generate the autoregulation status signal from the compliance value. The autoregulation status signal may be generated, at least in part, on the results from block 220, including whether the compliance value is similar to a known autoregulation parameter and/or whether the compliance value exceeds a threshold autoregulation value. The autoregulation status signal may represent the status of cerebral autoregulation of the patient, including an indication that cerebral autoregulation has become impaired.

At decision block 230, the controller 110 may determine whether cerebral autoregulation of the patient has become impaired. This decision may be based, at least in part, on the compliance value, the autoregulation status signal, or both. If cerebral autoregulation is determined to be impaired, the process 200 may continue at block 235. If not, the process 200 may continue at block 240.

At block 235, the controller 110 may generate an alarm signal that may bring the impaired autoregulation status of the patient to a treating physician's attention. The alarm signal, as previously discussed, may be used to generate a visual or audible alarm on the output device 115. The process 200 may continue at block 240 after the alarm signal has been generated.

At block 240, the controller 110 may transmit the autoregulation status signal to the output device 115. If no autoregulation impairment is determined at block 230, the autoregulation status signal may include information for display to the treating physician via the display device 120. A visual representation of the autoregulation status may be displayed. If cerebral autoregulation impairment is determined at block 230, the autoregulation status signal may be transmitted with the alarm signal or may itself act as the alarm signal, as previously described. The alarm signal may cause the output device 115 to present a visual indication of the autoregulation impairment via the display device 120 and possibly an audible indication of the autoregulation impairment via the audio device 125.

The process 200 may end after block 240.

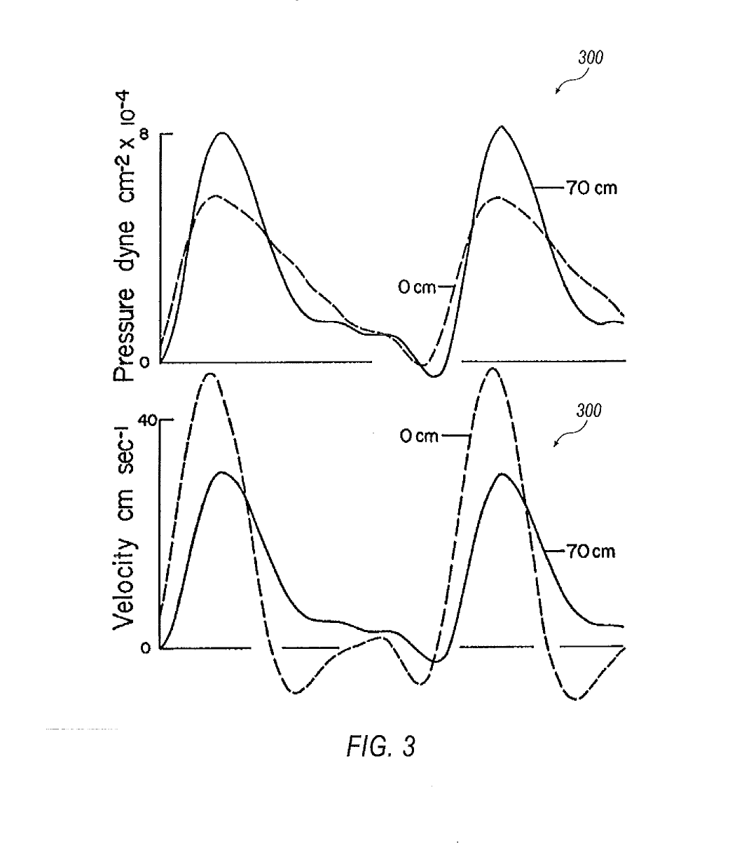

FIGS. 3-5 are charts of various characteristics of compliance. These characteristics may be used to develop the compliance metrics and/or compliance values previously discussed.

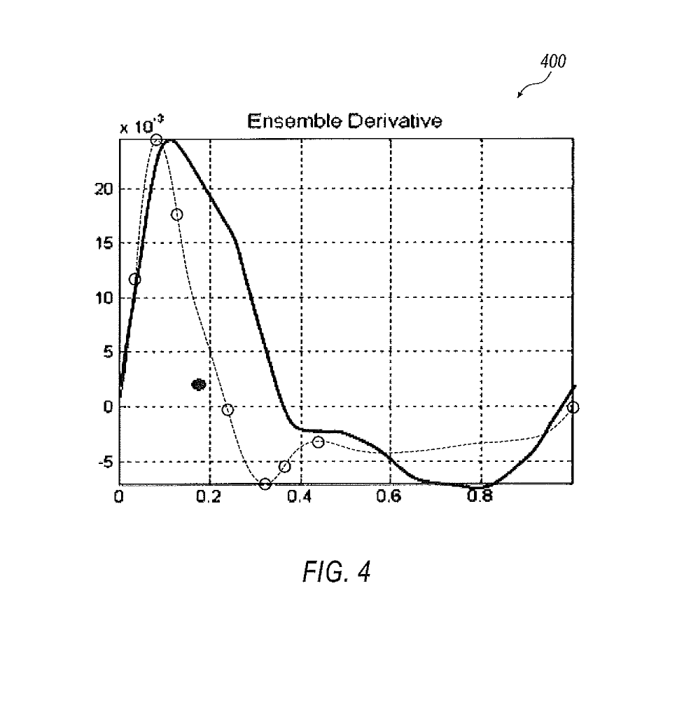

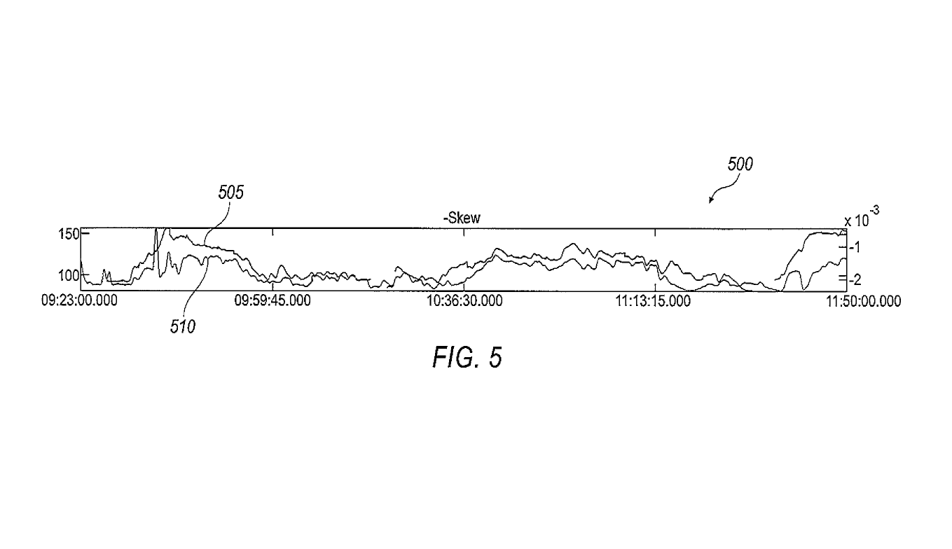

FIG. 3 is a chart showing examples of pressure and velocity pulses through a more compliant and less compliant blood vessel. See Barnard A C L, Hunt W A, Timlake W P, Varley E; ‘Peaking of the Pressure Pulse in Fluid Filled Tubes of Spatially Varying Compliance’; Biophysical Journal; Vol. 6; 1966. As illustrated in these charts, compliance affects both pressure and velocity. The top chart 300 in FIG. 3 illustrates how pressure changes over time in a more compliant blood vessel (dashed line) and in a less compliant blood vessel (solid line). The bottom chart 305 in FIG. 3 illustrates velocity changes over time in the more compliant blood vessel (dashed line) and the less compliant blood vessel (solid line). FIG. 4 is a chart 400 of an example ensemble average photoplethysmograph (PPG) derivative pulses and high and low arterial pressures normalized in height and pulse period. The solid line in FIG. 4 represents high pressure and low arterial compliance while the dashed line in FIG. 4 represents low pressure and high arterial compliance. FIG. 5 illustrates a chart 500 of example arterial pressure 505 with pulse derivative skew 510. As illustrated, the skew value trends with pressure, and thus, compliance. This skew metric, derived from the physiological signal, the photoplethysmogram, may be used to determine compliance changes in the vessels.

With regard to the processes, systems, methods, heuristics, etc. described herein, it should be understood that, although the steps of such processes, etc. have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claimed invention.

Accordingly, it is to be understood that the above description is intended to be illustrative and not restrictive. Many embodiments and applications other than the examples provided would be apparent upon reading the above description. The scope of the invention should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the invention is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

1. A system comprising:
a controller configured to receive a physiological signal representing a non-invasive measure of a physiological parameter of a patient, wherein the controller is configured to apply a compliance metric to the physiological signal and generate an autoregulation status signal that indicates a status of cerebral autoregulation in the patient, wherein the autoregulation status signal is based at least in part on the compliance metric applied to the physiological signal.

2. A system as set forth in claim 1, wherein the controller is configured to determine a compliance value after applying the compliance metric to the physiological signal and generate the autoregulation status signal based at least in part on the compliance value.

3. A system as set forth in claim 2, wherein the controller is configured to compare the compliance value to a plurality of known autoregulation parameters and generate the autoregulation status signal based at least in part on one of the known autoregulation parameters.

4. A system as set forth in claim 2, wherein the controller is configured to compare the compliance value to a threshold autoregulation value and generate the autoregulation status signal based at least in part on whether the compliance value exceeds the threshold autoregulation value.

5. A system as set forth in claim 2, wherein the controller is configured to determine the compliance value by calculating at least one of the following characteristics of the measured physiological parameter: mean, standard deviation, skew, kurtosis, pulse wave area, center of area, rotational moment, pulse period, and peak to peak amplitude.

6. A system as set forth in claim 1, further comprising a display device configured to receive the autoregulation status signal from the controller and generate a visual representation of the status of cerebral autoregulation of the patient.

7. A system as set forth in claim 1, wherein the controller is configured to generate an alarm signal if the autoregulation status signal represents impaired cerebral autoregulation.

8. A system as set forth in claim 1, further comprising a sensor configured to non-invasively measure a physiological parameter and generate a physiological signal, wherein the sensor includes a near-infrared spectroscopy sensor.

9. A method comprising:
receiving a physiological signal representing a measured physiological parameter;
applying a compliance metric to the received physiological signal; and
generating an autoregulation status signal that indicates a status of cerebral autoregulation in the patient, wherein the autoregulation status signal is based at least in part on the compliance metric applied to the physiological signal.

10. A method as set forth in claim 9, further comprising:
determining a compliance value after applying the compliance metric to the physiological signal, and
wherein generating the autoregulation status signal includes generating the autoregulation status signal based at least in part on the compliance value.

11. A method as set forth in claim 10, further comprising:
comparing the compliance value to a plurality of known autoregulation parameters, and
wherein generating the autoregulation status signal includes generating the autoregulation status signal based at least in part on one of the known autoregulation parameters.

12. A method as set forth in claim 10, further comprising:
comparing the compliance value to a threshold autoregulation value, and
wherein generating the autoregulation status signal includes generating the autoregulation status signal based at least in part on whether the compliance value exceeds the threshold autoregulation value.

13. A method as set forth in claim 10, further comprising determining the compliance value includes calculating at least one of the following characteristics of the measured physiological parameter: mean, standard deviation, skew, kurtosis, pulse wave area, center of area, rotational moment, pulse period, and peak to peak amplitude

14. A method as set forth in claim 9, further comprising:
outputting the autoregulation status signal to a display device, and
displaying a visual representation of the status of cerebral autoregulation of the patient in accordance with the autoregulation status signal on the display device.

15. A method as set forth in claim 9, further comprising:
determining whether the autoregulation status signal represents impaired cerebral autoregulation; and
generating an alarm signal if the autoregulation status signal represents impaired cerebral autoregulation.

16. A system comprising:
a sensor configured to non-invasively measure a physiological parameter by near-infrared spectroscopy and output a physiological signal representing the measured physiological parameter;
a controller in communication with the sensor and configured to apply a compliance metric to the physiological signal and generate an autoregulation status signal that indicates a status of cerebral autoregulation in the patient, wherein the autoregulation status signal is based at least in part on the compliance metric applied to the physiological signal; and
a display device configured to receive the autoregulation status signal from the controller and generate a visual representation of the status of cerebral autoregulation of the patient,
wherein the controller is configured to generate an alarm signal if the autoregulation status signal indicates that cerebral autoregulation of the patient is impaired.

17. A system as set forth in claim 16, wherein the controller is configured to determine a compliance value after applying the compliance metric to the physiological signal and generate the autoregulation status signal based at least in part on the compliance value.

18. A system as set forth in claim 17, wherein the controller is configured to compare the compliance value to a plurality of known autoregulation parameters and generate the autoregulation status signal based at least in part on one of the known autoregulation parameters.

19. A system as set forth in claim 17, wherein the controller is configured to compare the compliance value to a threshold autoregulation value and generate the autoregulation status signal based at least in part on whether the compliance value exceeds the threshold autoregulation value.

20. A system as set forth in claim 17, wherein the controller is configured to determine the compliance value by calculating at least one of the following characteristics of the measured physiological parameter: mean, standard deviation, skew, kurtosis, pulse wave area, center of area, rotational moment, pulse period, and peak to peak amplitude.
